(19) United States
(12) Patent Application Publication
(10) Pub. No.: US 2025/0249269 A1
(43) Pub. Date: Aug. 7, 2025

(54) RF ABLATION SYSTEM WITH FEEDBACK-BASED SPLIT-ELECTRODE GROUND PAD PLACEMENT

(71) Applicant: Boston Scientific Neuromodulation Corporation, Valencia, CA (US)

(72) Inventor: Thomas Warren Stouffer, Chatsworth, CA (US)

(21) Appl. No.: 19/044,033

(22) Filed: Feb. 3, 2025

Related U.S. Application Data
(60) Provisional application No. 63/549,805, filed on Feb. 5, 2024.

(51) Int. Cl. A61N 1/40; A61N 1/06; A61N 1/08

(52) U.S. Cl. CPC A61N 1/403; A61N 1/06; A61N 1/08

(57) ABSTRACT

An RF ablation system includes an RF electrode, a split-electrode ground pad, and an RF generator. The split-electrode ground pad includes a first ground pad, a second ground pad, and a ground-pad cable coupled to the first and second ground pads. The RF generator is configured to deliver RF energy from the RF generator through the RF electrode to patient tissue of a patient to cause ablation using the split-electrode ground pad as a counter-electrode. The RF generator includes ports, a display, a memory, and a processor. The processor is configured to execute instructions including: a) prior to delivery of the RF energy, determining a value indicative of an impedance between the first ground pad and the second ground pad; b) displaying, on the display, a representation of the first value; and c) repeating steps a) and b) until a termination condition is met.

### CROSS-REFERENCE TO RELATED APPLICATIONS

This application claims the benefit under 35 U.S.C. § 119 (e) of U.S. Provisional Patent Application Ser. No. 63/549,805, filed Feb. 5, 2024, which is incorporated herein by reference.

### FIELD

The present disclosure is directed to the area of radiofrequency (RF) ablation systems and methods of making and using the systems. The present disclosure is also directed to RF ablation systems and methods for feedback-based placement of a split-electrode ground pad, as well as methods of making and using the same.

### BACKGROUND

Radiofrequency (RF) generators and electrodes can be used for pain relief or functional modification. Radiofrequency ablation (RFA) is a safe, proven methodology of interrupting pain signals, such as those coming from irritated facet joints in the spine, genicular nerves in the knee, and femoral and obturator nerves in the hip. Radiofrequency current is used to heat up a small volume of nerve tissue, thereby interrupting pain signals from that specific area. Radiofrequency ablation is designed to provide long-lasting pain relief.

For example, an RF electrode can be positioned near target tissue and then used to heat the target tissue by RF power dissipation of the RF signal output in the target tissue.

### BRIEF SUMMARY

One aspect is a radiofrequency (RF) ablation system that includes an RF electrode having an electrode element and an RF electrode cable electrically coupled to the electrode element; a split-electrode ground pad having a first ground pad, a second ground pad, and a ground-pad cable electrically coupled to the first and second ground pads, wherein the ground-pad cable includes at least one first conductor electrically coupled to the first ground pad and at least one second conductor electrically coupled to the second ground pad; and an RF generator coupleable to the RF electrode and the split-electrode ground pad, the RF generator being configured to deliver RF energy from the RF generator through the RF electrode to patient tissue of a patient to cause ablation using the split-electrode ground pad as a counter-electrode. The RF generator includes a plurality of ports configured to individually couple the RF electrode cable and the split-electrode ground pad cable to the RF generator, a display, a non-transitory memory having processor-executable instructions stored thereon, and a processor coupled to the non-transitory memory and configured to execute the processor-executable instructions. The processor-executable instructions include a) prior to delivery of the RF energy, determining a first value that is indicative of an impedance between the first ground pad and the second ground pad, b) displaying, on the display, a representation of the first value, and c) repeating steps a) and b) until a termination condition is met.

Another aspect is a method that includes placing a split-electrode ground pad in contact with a patient, wherein the split-electrode ground pad includes a first ground pad, a second ground pad; coupling the split-electrode ground pad to an RF generator; until a termination condition is met, repeatedly performing the following steps: (a) determining, by the RF generator, a first value that is indicative of an impedance between the first ground pad and the second ground pad, and (b) displaying, on a display of the RF generator, a representation of the first value; and subsequent to meeting the termination condition, delivering RF energy from the RF generator to an RF electrode, using the split-electrode ground pad as a counter-electrode, to ablate patient tissue.

A further aspect is a processor-readable storage medium, having stored thereon processor-executable code that, upon execution by at least one processor, enables actions, including: until a termination condition is met, repeatedly performing the following steps: (a) determining a first value that is indicative of an impedance between a first ground pad and a second ground pad of a split-electrode ground pad, and (b) displaying a representation of the first value; and subsequent to meeting the termination condition, delivering RF energy from an RF generator to an RF electrode, using the split-electrode ground pad as a counter-electrode, to ablate patient tissue.

In at least some embodiments, the first value indicative of the impedance is the impedance. In at least some embodiments, the first value indicative of the impedance is a current, a voltage, or an impedance differential (for example, a difference between the measured or determined impedance and a reference impedance).

In at least some aspects, the termination condition includes at least one of: a user command, an initiation of the delivery of the RF energy, a determination of the first value reaching a threshold value, a determination of the first value reaching a predetermined range, or a determination that a predetermined period of time has occurred since the split-electrode ground pad has last moved. In at least some aspects, the representation of the first value is a display of the first value, a graph of the first value versus time, a histogram of the first value, a time-series of histograms of the first value, a moving slider that indicates the first value, or one or more lights indicating a threshold or range of the first value. In at least some aspects, the repeating includes repeating steps a) and b) at least once per second.

In at least some aspects, determining the first value includes applying a voltage between the first ground pad and the second ground pad. In at least some aspects, an area of the first ground pad is at least 10 square centimeters and an area of the second ground pad is at least 10 square centimeters. In at least some aspects, the displaying includes displaying the representation using color, shading, pattern, size, text, shape, or any combination thereof to identify each of a plurality of different ranges of the first value.

In at least some aspects, the instructions or the method further include(s) providing at least one of a visual message or an auditory message for at least one of the following conditions: 1) the first value is above a first threshold value or 2) the first value is below a second threshold value. In at least some aspects, the instructions or the method further include(s) initiating an alarm if the first value is outside of a predetermined range. In at least some aspects, the instructions or the method further include(s) initiating the determining when an associated user command is received or when the ground pad cable is received by one of the ports of the RF generator.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, in which:

FIG. 1 is a schematic side view of components of one embodiment of an RF ablation system;

FIG. 2 is a block diagram of another embodiment of an RF ablation system;

FIG. 3 is a block diagram of an embodiment of the RF ablation system of FIG. 2 being applied to a patient;

FIG. 4 is a front view of one embodiment of a screen that may be used in an embodiment of the display of FIG. 2; and

FIG. 5 is a flowchart of one embodiment of a method of using an embodiment of an RF ablation system.

### DETAILED DESCRIPTION

The present disclosure is directed to the area of radiofrequency (RF) ablation systems and methods of making and using the systems. The present disclosure is also directed to RF ablation systems and methods for feedback-based placement of a split-electrode ground pad, as well as methods of making and using the same.

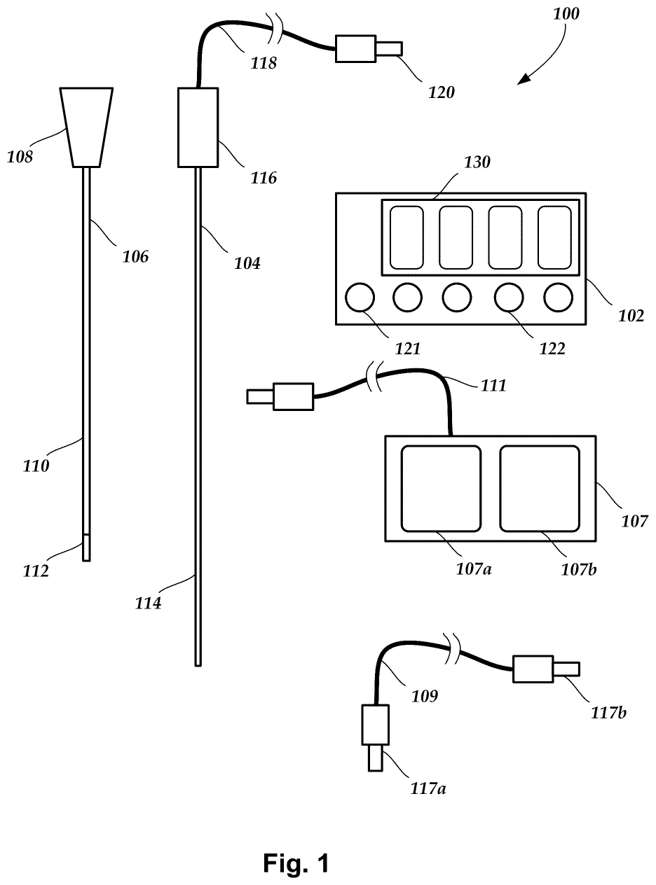

FIG. 1 illustrates one embodiment of an RF ablation system 100 that includes an RF generator 102, an RF electrode 104, a cannula 106, a ground pad 107, and an optional extension cable 109. In at least some embodiments, the cannula 106 includes a cannula hub 108, an insulated shaft 110, and an active tip 112. In at least some embodiments, the insulated shaft 110 is hollow for receiving the RF electrode 104. When inserted, in at least some embodiments, the RF electrode 104 contacts, and energizes, the active tip 112 of the cannula 106 to produce RF ablation.

In at least some embodiments, the RF electrode 104 includes an electrode shaft 114, an electrode hub 116, a cable 118 that is electrically coupled to the electrode shaft 114, and a connector 120 for connecting to an electrode port 122 of the RF generator 102 to energize the electrode shaft 114 via the cable 118 and connector 120. In at least some embodiments, the optional adapter or extension 109 includes a cable 119 and connectors 117a, 117b for coupling the RF electrode 104 to the RF generator 102. It will be recognized that some embodiments of RF ablation systems utilize the RF electrode 104 for ablation instead of, or in addition to, the cannula 106.

The RF generator 102 includes one or more electrode ports 122, at least one ground port 121, and at least one display 130. In at least some embodiments, each electrode port 122 is associated with a portion of the display 130 (or a different screen) and can receive the connector 120 from an RF electrode 104. In various embodiments, information such as current, voltage, status, or the like or any combination thereof can be displayed on the display 130. In at least some embodiments, each electrode port 122 corresponds to an independent channel for operating a RF electrode 104.

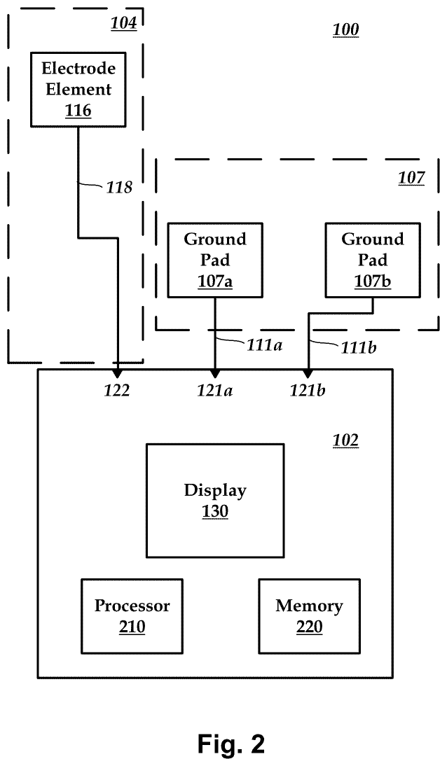

The RF generator 102 includes at least one ground port 121 for attachment of the ground pad 107 via at least one ground pad cable 111. The ground pad 107 is a split-electrode ground pad that includes a first ground pad 107a and a second ground pad 107b. One non-limiting example of a split-electrode ground pad 107 includes a substrate, two conductive electrodes (e.g., first and second ground pads 107a, 107b) disposed on the substrate, and an adhesive (in at least some embodiments, a conductive adhesive) for attachment of the split-electrode ground pad to the skin of the patient with the two conductive electrodes in electrically conductive contact with the skin. In at least some embodiments, the RF generator has a single ground port for the split-electrode ground pad 107, as illustrated in FIG. 1. In other embodiments, the RF generator has separate corresponding ground ports for each of the first and second ground pads 107a, 107b (see, FIG. 2).

Examples of RF generators and RF ablation systems and methods of making and using the RF generators and RF ablation systems can be found at, for example, U.S. Pat. Nos. 9,717,552; 9,956,032; 10,111,703; 10,136,937; 10,136,942; 10,136,943; 10,194,971; 10,342,606; 10,363,063; 10,588,687; 10,631,915; 10,639,098; 10,639,101; 11,058,455; and 11,633,226 and U.S. Patent Application Publications Nos. 2014/0066917; 2014/081260; 2014/0121658; 2017/0050038; 2021/0236191; 2022/0023588; 2022/0202484; 2022/0202485; and 2022/0226039, all of which are incorporated herein by reference in their entireties.

FIG. 2 is a block diagram of another embodiment of an RF ablation system 100. The RF ablation system 100 includes an RF generator 102, an RF electrode 104, and a split-electrode ground pad 107. The RF electrode 104 includes an electrode element 116 and an RF electrode cable 118 with the RF electrode cable 118 electrically coupled to the electrode element 116. The split-electrode ground pad 107 includes a first ground pad 107a, a second ground pad 107b, and at least one ground-pad cable with first and second ground pad conductors 111a, 111b. The RF generator 102 includes a display 130, a non-transitory memory 220, a processor 210, and multiple ports. For instance, the ports on RF generator 102 can include a ground port 121a, a ground port 121b, and an electrode port 122.

Any suitable non-transitory memory 220 can be used including, but not limited to, a volatile memory, a semi-volatile memory, a random access memory, a static memory, or any other suitable non-transitory storage medium. Any suitable processor 210 can be used including, but not limited to, include a microprocessor, a microcontroller, a graphics processor, a coprocessor, a field-programmable gate array, a programmable logic device, a signal processor, or any other circuit suitable for processing data.

The split-electrode ground pad 107 includes at least two ground pads (e.g., first ground pad 107a and second ground pad 107b) that provide a relatively large area of contact (for example, compared to the active surface of the RF electrode or cannula) with the patient. In the illustrated embodiment of FIG. 2, the RF electrode 104 is coupled to the RF generator 102 through the electrode port 122, the ground pad 107a is coupled through the port 121a, and the ground pad 107b is coupled through the port 121b. (In other embodiments, the ground pads 107a, 107b are coupled to a single ground port 121, as illustrated in FIG. 1.) The RF generator 102 is configured to deliver RF energy through the RF electrode 104 to ablate, or otherwise provide therapy to, patent tissue. The delivery of the RF energy to target patent tissue causes ablation using the split-electrode ground pad 107 as a counter-electrode, ground electrode, neutral electrode, or return electrode, closing the electrical circuit formed with the RF electrode 104.

During RF ablation, the RF current can be thought of as flowing from the RF electrode 104 through the body of the patient back to the RF generator 102 through the ground pad 107. In at least some embodiments, during RF ablation, the target patient tissue is heated and ablated (e.g., destroyed) to achieve a specific therapeutic effect, such as pain relief or any other suitable therapeutic effect. The area of the split-electrode ground pad 107 is significantly larger than the area of RF electrode 104 and is intended to prevent, or resist, burning or otherwise damaging the tissue or skin to which the split-electrode ground pad 107 is attached. For instance, in at least some embodiments, each of the ground pads (e.g., the ground pads 107A and 107B) of the split-electrode ground pad 107 has a surface area of at least 10, 25, 40, 50, 100, 150, 170 or more square centimeters, although any other suitable surface area can be used.

It is found, however, when the split-electrode ground pad (or any other ground pad) is not fully or correctly attached to the skin of the patient or the adhesive of the ground pad is degraded or other problem or defect, the skin or other tissue of the patient can be burned or otherwise damaged. As described herein, the RF ablation system 100 can monitor the attachment of the split-electrode ground pad 107 to the patient to facilitate or assist the clinician, operator, or other individual to adequately, fully, or correctly attach the split-electrode ground pad to the patient. In at least some embodiments, the monitoring can be useful, for example, to prevent or reduce burning or other damage to the skin or other tissue of the patient.

The RF ablation system 100 measures or otherwise determines the impedance between the two ground pads 107a, 107b of the split-electrode ground pad 107 as the split-electrode ground pad is being attached to the patient prior to delivering RF for ablation. Alternatively or additionally, the RF ablation system 100 can measure or otherwise determine a value that is indicative of the impedance, such as a current or voltage between the two ground pads 107a, 107b. The measurement or determination of impedance is discussed herein, but it will be understood that such a measurement or determination can be replaced with measurement or determination of a value that is indicative of impedance.

In at least some embodiments, the measurement or determination of the impedance is performed repeatedly, continuously, or periodically as the split-electrode ground pad is being attached to the patient prior to delivering RF for ablation. In at least some embodiments, the measurement or determination of the impedance is performed in real-time as the split-electrode ground pad is being attached to the patient prior to delivering RF for ablation. A representation of the measured or determined impedance is displayed on a display 130 of the RF generator 102 so that the clinician or other individual can assess the quality of the attachment of the split-electrode ground pad 107 to the patient during attachment.

The impedance is expected to decrease as the two ground pads 107a, 107b are more fully or correctly attached to the patient. In at least some embodiments, the RF generator 102 can indicate on the display 130 when the impedance reaches a predetermined impedance range or is below a predetermined impedance threshold value that indicates full, adequate, or correct attachment of the split-electrode ground pad 107 to the patient. In at least some embodiments, a relatively high impedance can indicate a variety of issues relative to the attachment of the split-electrode ground pad 107 including incorrect or partial attachment, degraded or inadequate adhesive on the split-electrode ground pad, or some other defect or problem, or any combination thereof. In at least some embodiments, an impedance that is too small may indicate a short between the two ground pads 107a, 107b, or some other defect or problem.

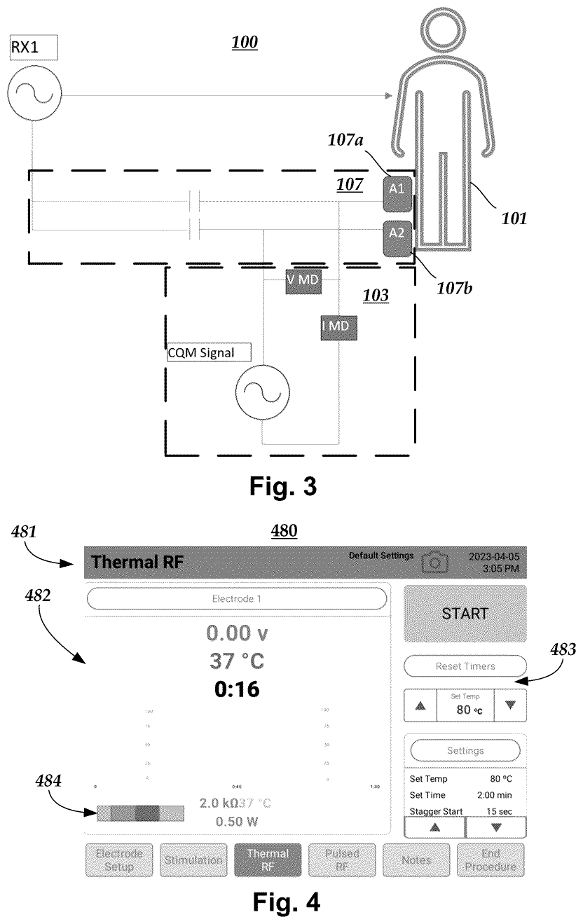

Any suitable method can be used to measure impedance. As an example, FIG. 3 illustrates one arrangement for measurement of impedance between two ground pads 107a, 107b of a split-electrode ground pad 107 applied to a (human) patient 101. The ground pad 107A and the ground pad 107B are attached to, or otherwise placed in contact with, the patient 101. During ablation, a signal RX1 is applied by the RF generator 102 to the RF electrode 104 (not shown in FIG. 3) using the split-electrode ground pad 107 as the counter-electrode, neutral electrode, ground electrode, or return electrode.

With respect to FIG. 3, in at least some embodiments, prior to ablation, the RF generator 102 applies a relatively small voltage VMD between ground pad 107A and ground pad 107B using a measurement circuit 103 of the RF generator 102 (or other device). In at least some embodiments, the measurement circuit 103 is a high-resistance, low-noise, high-resolution measurement circuit. In at least some embodiments, the applied voltage is part of a Contact Quality Monitor (CQM) signal that is applied between the ground pad 107A and the ground pad 107B. In at least some embodiments, the measurement circuit 103 measures a current IMD between the ground pad 107A and the ground pad 107B. The current IMD and voltage VMD can be used to determine the impedance between the ground pad 107A and the ground pad 107B. The impedance can be the magnitude of the impedance or the complex impedance.

In at least some embodiments, the RF ablation system 100 or RF generator 102 includes a set-up mode that includes attachment of the split-electrode ground pad 107 to the RF generator and to the patient 101. Such a set-up mode is described below but it will be understood, that, in other embodiments, there is no specific set-up mode and the attachment of the split-electrode ground pad 107 to the RF generator and to the patient is part of the operation of the RF ablation system 100. Accordingly, the description herein also applies to RF ablation systems 100 and RF generators 102 that do not include a set-up mode in all details other than the explicit reference to the set-up mode.

In a set-up mode (or any other suitable mode, procedure, operation, or method) that occurs before the RF ablation, a clinician, operator, or other individual sets up the equipment for ablation including attaching the split-electrode ground pad 107 to the patient. During the set-up mode (or any other suitable mode, procedure, operation, or method) as the clinician, operator, or other individual is attaching the split-electrode ground pad 107 to the patient, the RF generator 102 displays one or more representations of the impedance (or a value that is indicative of the impedance, such as a voltage, a current, an impedance differential, or the like) between ground pad 107A and ground pad 107B to assist the clinician or other individual with the adequate, full, or correct attachment of the split-electrode ground pad to the patient. The representation (or any other indicator) can provide feedback so that the clinician, operator, or other individual can adequately, fully, or correctly attach the split-electrode ground pad 107 to the patient.

The representation displayed by the RF generator 102 includes one or more visual indicators (e.g., graphical representations) that indicate or otherwise represent impedance or a value indicative of the impedance between the ground pad 107A and the ground pad 107B. Non-limiting examples of the visual indicators, that can convey the value, approximate value (including, for example, indicating that the impedance is within a predefined range or above or below or equal to a threshold), or relative value (e.g., a marker between two endpoints) of the impedance, include text (for example, a value of the impedance), a dial, a bar graph, a time-series of bar graphs, an icon that indicates whether a suitable impedance has been reached, a histogram, a time-series of histograms, a moving slider, a graph of impedance versus time, one or more lights indicating impedance thresholds or ranges, or any other suitable visual indicator that can convey the value, approximate value, or relative value of the impedance. Instead of impedance, the visual indicator can convey a value that is indicative of impedance, such as, for example, an impedance differential using a reference impedance, a current (e.g., the current IMD between the ground pad 107A and the ground pad 107B), a voltage (e.g., the voltage VMD between the ground pad 107A and the ground pad 107B) or any other suitable value that is mathematically related to the impedance between the ground pad 107A and the ground pad 107B.

In at least some embodiments, the representation displayed by the RF generator 102 also includes one or more indicators of a variance that is associated with the impedance (or the value indicative of impedance) between the ground pad 107A and the ground pad 107B. For instance, in at least some embodiments, the RF generator determines a variance of the impedance between the ground pad 107A and the ground pad 107B over a particular time interval, such as over 1, 2, 5, or 10 seconds or over any another suitable time interval. A low variance as the ground pads 107a, 107b are being placed and adjusted can indicate that the ground pads 107a, 107b are correctly or fully positioned on the patient.

FIG. 4 illustrates one embodiment of a visual indicator 484 that has the form of a moving slider or bar graph. As another example, an icon that indicates whether a suitable impedance has been reached may include, for example, an icon that changes to a particular color when a suitable impedance has been reached, an icon that appears when a suitable impedance has been reached, and icon that changes shapes when a suitable impedance has been reached, or the like. In at least some embodiments, one or more visual indicators can indicate the impedance value, approximate value, or relative value or other information that is associated with the impedance using color, shading, pattern, size, shape, or any other suitable visual parameters.

In at least some embodiments, at least one of the visual indicators indicates whether the impedance between ground pad 107A and ground pad 107B is in a predetermined range of impedance that indicates adequate, correct, or full attachment of the split-electrode ground pad 107 to the patient. As an example, the predetermined range may be between a lower value, such as 0.25, 0.4, 0.5, 1, 5, 10, or 15 Ohms, and an upper value, such as 30, 50, 80, 100, 150, 200, or 250 ohms s or any other suitable range, which may depend, for example, on the RF ablation system, the configuration or type of the split-electrode ground pad, the adhesive, or the like or any combination thereof. Any of these predetermined ranges can be inclusive of one or both endpoints or exclusive of both endpoints. In at least some embodiments, at least one of the visual indicators indicates whether the impedance between the ground pad 107A and the ground pad 107B is below (or below or equal to) a first threshold value of impedance that indicates adequate, correct, or full attachment of the split-electrode ground pad 107 to the patient. As an example, the first threshold value may be 250, 150, 100, 80, 50, or 30 Ohms or any other suitable value.

In at least some embodiments, at least one of the visual indicators indicate whether the impedance between ground pad 107A and ground pad 107B is below (or below or equal to) a second threshold value of impedance (or below the predetermined range of impedance) that indicates a short or other defect that produces an impedance that is lower than expected. As an example, the second threshold value may be 15, 10, 5, 1, 0.5, 0.4, or 0.25 Ohms or any other suitable value. An example of another defect that may produce low (or high) impedance is when an operator erroneously connects an unsupported component, such as a single electrode pad or other component that is not a split-electrode ground pad.

In at least some embodiments, at least one of the visual indicators indicates whether the impedance between ground pad 107A and ground pad 107B is above (or above or equal to) that first threshold value of impedance (or above the predetermined range of impedance) that indicates that the split-electrode ground pad 170 is not adequately, fully, or correctly attached to the patient. For example, when the impedance is greater than the predetermined range (for example using the range recited above, greater than about 250, 200, 150, 100, 80, 50, or 30 Ohms), the visual indicator indicates that the split-electrode ground pad 107 is not attached to a patient adequately, correctly, or fully or that the adhesive is degraded.

In at least some embodiments, for an impedance that is not in the predetermined range or not below or equal to the first threshold value (or not above or equal to the second threshold value) the system may also provide an error message, an alarm, a warning, or any other suitable visual or auditory indication that the impedance is too high (or too low). In at least some embodiments, this may occur, in particular, when the set-up mode is ended or when the clinician or other individual directs the RF generator 102 to apply an ablation current or voltage. For instance, in at least some embodiments, the RF generator 102 generates an audible alarm if the impedance value is too high (or too low). In at least some embodiments, the display 130 may provide additional information as to the value of the impedance or other aspects that relate to the issue that is causing the impedance to be outside of the normal range. In at least some embodiments, the RF ablation system 100 or RF generator 102 may prevent the generation of RF ablation energy if the impedance value is too high (or, in some embodiments, too low).

Additionally or alternatively, one or more auditory indicators can be presented to provide similar information as the visual indicator(s) to the clinician, operator, or other individual such, as for example, an auditory indicator that is presented when the impedance is too low or too high or an auditory indicator that is presented when the impedance is within a predetermined range or below a threshold value or any combination thereof. For instance, tone, pitch, frequency, or other aspects of a sound emitted by the RF generator 102 may vary based on the impedance between the ground pad 107A and the ground pad 107B. For example, in at least some embodiments, a tone may get louder, higher in pitch, or vary in another suitable manner as the impedance increases, or vice versa. In other embodiments, a particular sound is provided or ceases when a suitable impedance is reached. In other embodiments, RF generator may, via computer-generated speech, state that a suitable impedance has been reached.

The clinician, operator, or other individual can use feedback from the visual or auditory indicator(s) to assist in attachment of the split-electrode ground pad 107 to the patient. In at least some embodiments, the visual or auditory indicator(s) may provide feedback that assists the clinician or other individual in achieving an impedance within the predetermined expected range or at or below the first threshold value (or at or above the second threshold value or any combination thereof). One or more of the visual indicators, as well as other information useful to the operator, are provided on display 130.

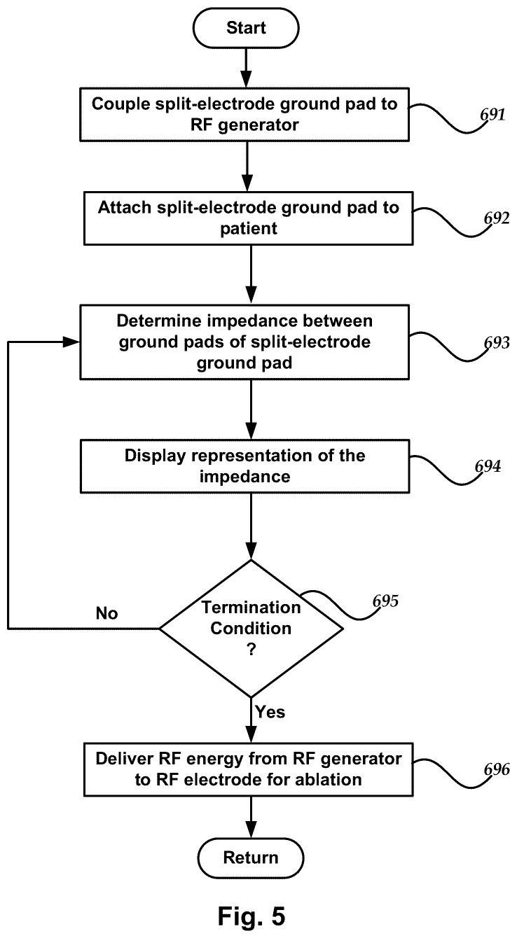

Attachment of the split-electrode ground pad 107 to the patient occurs prior to RF ablation in which RF energy is delivered to the target patient tissue. FIG. 5 is a flowchart of one embodiment of a method for ablating tissue of a patient. The method can be performed using an RF ablation system 100 with the RF ablation system 100 performing at least some of the steps of the method.

In step 691, a split-electrode ground pad 107 is coupled to an RF generator 102. In step 692, the split-electrode ground pad is attached to the patient or otherwise placed in contact with a patient.

In step 693 (and prior to RF ablation (step 696)), an impedance is measured or otherwise determined between the first ground pad (e.g., the ground pad 107A) and the second ground pad (e.g., the ground pad 107B). In step 694, a representation of the impedance is displayed on a display 130 of the RF generator 102. In at least some embodiments, the process then proceeds to decision block 695 in which a determination is made as to whether a termination condition has been met. When the determination at decision block 695 is negative, the process returns to step 693. In at least some embodiments, the repeating of the steps 693 to 695 occurs in a continuous manner or in a periodic manner, such as at least once per second, at least twice per second, at least once per two seconds, or any another suitable time frequency or pattern.

When the determination is positive, in at least some embodiments, the process then continues to step 696. In step 696, which is subsequent to the termination condition, RF energy is delivered from the RF generator to an RF electrode, using the split-electrode ground pad as a counter-electrode, ground electrode, return electrode, or neutral electrode, to ablate patient tissue.

Any suitable termination condition or set of termination conditions can be used in step 696. In at least some embodiments, there can be multiple termination conditions that can be met and, in step 696, the determination is whether any one (or more than one) of the termination conditions have been met. In at least some embodiments, one termination condition is a determination that the impedance is within the predetermined range or below (or below or equal to) the first threshold value, thereby indicating that the split-electrode ground pad 170 is adequately or fully attached to the patient. In at least some embodiments, one termination condition is an action by the clinician, operator, or other individual, such as by the clinician, operator, or other individual selecting on option or control on the RF generator 102 indicating that placement of the split-electrode ground pad 107 is complete. In at least some embodiments, one termination condition is a determination by the RF generator 102 that a predetermined period of time (e.g., at least five seconds, or another suitable period of time) has occurred since either the ground pad 107A or the ground pad 107B has been moved. In at least some embodiments, the termination condition is the beginning of the delivery of RF energy via the RF electrode 104 or is a command to begin the delivery of RF energy via the RF electrode 104, or the like.

In at least some embodiments, the RF generator 102 provides a visual or auditory message when a termination condition has occurred-such as when the impedance is in the predetermined impedance range, when split-electrode ground pad has not been moved for the predetermined period of time, or the like.

In at least some embodiments, RF generator 102 prevents the delivery of RF energy under certain conditions, such as when the impedance is outside of the predetermined impedance range or above (or above or equal to) the first threshold or below (or below or equal to) the second threshold. For instance, in at least some embodiments, RF generator 102 will not initiate the delivery of RF energy when the impedance measurement or determination indicates that the split-electrode ground pad is not adequately, correctly, or fully attached to the patient. In at least some embodiments, the RF generator 102 ceases delivery of RF energy and initiates an alarm, a warning, error message, or the like when the impedance measurement is not within the predetermined impedance range or below (or below or equal to) the first threshold.

FIG. 4 illustrates a front view of one embodiment of a display 480 of the RF generator 102. FIG. 4 illustrates a specific embodiment of the display 480 by way of example only. Any other suitable display for presenting information and, optionally, enabling selections may be used. In at least some embodiments, the display 480 includes a header 481, an information section 482, an RF ablation settings section 483, and a menu 484.

In at least some embodiments, the menu 484 displays selectable menu options. In at least some embodiments, the display 480 is a touch screen, and various options, including the selectable menu options in the menu 484, are selectable by touching the corresponding option on the display 480. In other embodiments, selections may be selected in other ways, such as one or more dials or buttons or the like on the RF generator 102, a trackpad, a track ball, a mouse, a keyboard, speech commands, physical gestures detected with a camera, or the like.

In at least some embodiments, the information section 483 shows information that is associated with the currently selected menu option. The type of information shown in some embodiments of the information section 483 varies based on the currently selected menu option. For instance, in the embodiment shown in FIG. 4, “Thermal RF” is the currently selected menu option, and the information section 483 includes information associated with one or more of the electrodes, such as the RF electrode 104, the split-electrode ground pad 107, or another suitable electrode.

In at least some embodiments, the header 481 includes various basic information, such as the currently selected menu option, various settings or other options selected by the user, time, date, or other suitable information. In at least some embodiments, the RF ablation settings section 483 includes various information associated with the RF ablation including the representation 485 of the impedance. For instance, the RF ablation settings sections 483 may include selectable RF ablation settings such as a selectable temperature to be used in the RF ablation, staggered start settings, settings for one or more timers, and a start/stop button that may be used to start or stop RF ablations. In at least some embodiments, the settings provided in RF ablation settings section 483 varies depending on the currently selected menu option.

As discussed above, before RF ablation begins, the clinician, operator, or other individual attaches the split-electrode ground pad 107 to the patient so that a large area of split-electrode ground pad 107 is in contact with the patient. The clinician, operator, or other individual can use feedback from the representation 485 of the impedance (and optionally other information such as auditory information and other visual information) to assist the operator in attachment of the split-electrode ground pad 107. Before or after attachment of the split-electrode ground pad 107, the clinician, operator, or other individual can use the RF ablation settings section 483 to input or alter settings for the RF ablation. Subsequently, the clinician, operator, or other individual can perform the RF ablation.

In at least some embodiments, the RF generator 102 may perform additional steps associated with beginning or ending the set-up mode or any other initial procedure, beginning or ending the RF ablation, or the like. For instance, in some embodiments, the RF generator 102 determines whether a cable has been received by one or the ports, whether a particular command has been received, or the like.

In at least some embodiments, a set-up mode (or steps 693-695 of FIG. 5) begins automatically when the ground pad cable 111 is connected to the RF generator 102. In at least some embodiments, a set-up mode (or steps 693-695 of FIG. 5) does not begin until that connection has been made and a suitable command has been input by the clinician, operator, or other individual via the RF generator 102. Similarly, in some embodiments, RF ablation does not begin until the proper connections have been made to the RF generator 102, the impedance is determined to be suitable, suitable settings for the RF ablation have been entered on the RF generator 102, and a command to begin RF ablation has been made by the operator via the RF generator 102.

The methods and systems described herein may be embodied in many different forms and should not be construed as limited to the embodiments set forth herein. Accordingly, the methods and systems described herein may take the form of an entirely hardware embodiment, an entirely software embodiment or an embodiment combining software and hardware aspects. Systems referenced herein typically include memory and typically include methods for communication with other devices including mobile devices. Methods of communication can include both wired and wireless (for example, RF, optical, or infrared) communications methods and such methods provide another type of computer readable media; namely communication media. Wired communication can include communication over a twisted pair, coaxial cable, fiber optics, wave guides, or the like, or any combination thereof. Wireless communication can include RF, infrared, acoustic, near field communication, Bluetooth™, or the like, or any combination thereof.

It will be understood that each block of the flowcharts, and combinations of blocks in the flowcharts and methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification and examples provide a description of the manufacture and use of the invention. Since many embodiments of the invention can be made without departing from the spirit and scope of the invention, the invention also resides in the claims hereinafter appended.

What is claimed is:

1. A radiofrequency (RF) ablation system, comprising:
an RF electrode comprising an electrode element and an RF electrode cable electrically coupled to the electrode element;
a split-electrode ground pad comprising a first ground pad, a second ground pad, and a ground-pad cable electrically coupled to the first and second ground pads, wherein the ground-pad cable comprises at least one first conductor electrically coupled to the first ground pad and at least one second conductor electrically coupled to the second ground pad; and
an RF generator coupleable to the RF electrode and the split-electrode ground pad, the RF generator being configured to deliver RF energy from the RF generator through the RF electrode to patient tissue of a patient to cause ablation using the split-electrode ground pad as a counter-electrode, the RF generator comprising
a plurality of ports configured to individually couple the RF electrode cable and the split-electrode ground pad cable to the RF generator,
a display,
a non-transitory memory having processor-executable instructions stored thereon, and
a processor coupled to the non-transitory memory and configured to execute the processor-executable instructions, the processor-executable instructions comprising
a) prior to delivery of the RF energy, determining a first value that is indicative of an impedance between the first ground pad and the second ground pad,
b) displaying, on the display, a representation of the first value, and
c) repeating steps a) and b) until a termination condition is met.

2. The RF ablation system of claim 1, wherein the first value indicative of the impedance is the impedance.

3. The RF ablation system of claim 1, wherein the first value indicative of the impedance is a current, a voltage, or an impedance differential.

4. The RF ablation system of claim 1, wherein the termination condition comprises at least one of: a user command, an initiation of the delivery of the RF energy, a determination of the first value reaching a threshold value, a determination of the first value reaching a predetermined range, or a determination that a predetermined period of time has occurred since the split-electrode ground pad has last moved.

5. The RF ablation system of claim 1, wherein the representation of the first value is a display of the first value, a graph of the first value versus time, a histogram of the first value, a time-series of histograms of the first value, a moving slider that indicates the first value, or one or more lights indicating a threshold or range of the first value.

6. The RF ablation system of claim 1, wherein the repeating comprises repeating steps a) and b) at least once per second.

7. The RF ablation system of claim 1, wherein determining the first value comprises applying a voltage between the first ground pad and the second ground pad.

8. The RF ablation system of claim 1, wherein an area of the first ground pad is at least 10 square centimeters and an area of the second ground pad is at least 10 square centimeters.

9. The RF ablation system of claim 1, wherein the displaying comprises displaying the representation using color, shading, pattern, size, text, shape, or any combination thereof to identify each of a plurality of different ranges of the first value.

10. The RF ablation system of claim 1, wherein the instructions further comprise providing at least one of a visual message or an auditory message for at least one of the following conditions: 1) the first value is above a first threshold value or 2) the first value is below a second threshold value.

11. The RF ablation system of claim 1, wherein the instructions further comprise initiating an alarm if the first value is outside of a predetermined range.

12. The RF ablation system of claim 1, wherein the instructions further comprise initiating the determining when an associated user command is received or when the ground pad cable is received by one of the ports of the RF generator.

13. A method, comprising:
coupling a split-electrode ground pad to an RF generator, wherein the split-electrode ground pad comprises a first ground pad, a second ground pad;
attaching the split-electrode ground pad to a patient;
until a termination condition is met, repeatedly performing the following steps:
(a) determining, by the RF generator, a value indicative of an impedance between the first ground pad and the second ground pad, and
(b) displaying, on a display of the RF generator, a representation of the first value; and
subsequent to meeting the termination condition, delivering RF energy from the RF generator to an RF electrode, using the split-electrode ground pad as a counter-electrode, to ablate patient tissue.

14. The method of claim 13, wherein the termination condition comprises at least one of: a user command, an initiation of the delivery of the RF energy, a determination of the first value reaching a threshold value, a determination of the first value reaching a predetermined range, or a determination that a predetermined period of time has occurred since the split-electrode ground pad has last moved.

15. The method of claim 13, wherein the representation of the first value is a display of the first value, a graph of the first value versus time, a histogram of the first value, a time-series of histograms of the first value, a moving slider that indicates the first value, or one or more lights indicating a threshold or range of the first value.

16. The method of claim 13, wherein the displaying comprises displaying the representation using color, shading, pattern, size, text, shape, or any combination thereof to identify each of a plurality of different ranges of the first value.

17. The method of claim 13, further comprising a) providing at least one of a visual message or an auditory message for at least one of the following conditions: 1) the first value is above a first threshold value or 2) the first value is below a second threshold value or b) initiating an alarm if the first value is outside of a predetermined range.

18. A processor-readable storage medium, having stored thereon processor-executable code that, upon execution by at least one processor, enables actions, comprising:
until a termination condition is met, repeatedly performing the following steps:
(a) determining a value that is indicative of an impedance between a first ground pad and a second ground pad of a split-electrode ground pad, and
(b) displaying a representation of the first value; and
subsequent to meeting the termination condition, delivering RF energy from an RF generator to an RF electrode, using the split-electrode ground pad as a counter-electrode, to ablate patient tissue.

19. The processor-readable storage medium of claim 18, wherein the termination condition comprises at least one of: a user command, an initiation of the delivery of the RF energy, a determination of the first value reaching a threshold value, a determination of the first value reaching a predetermined range, or a determination that a predetermined period of time has occurred since the split-electrode ground pad has last moved.

20. The processor-readable storage medium of claim 18, wherein the representation of the first value is a display of the first value, a graph of the first value versus time, a histogram of the first value, a time-series of histograms of the first value, a moving slider that indicates the first value, or one or more lights indicating a threshold or range of the first value.
